(19) United States
(12) Patent Application Publication
(10) Pub. No.: US 2026/0283655 A1
(43) Pub. Date: Sep. 24, 2026

(54) SCREW IMPLANTATION DEVICE

(71) Applicant: Point Robotics Medtech Inc., Hsinchu County (TW)

(72) Inventors: SHIH-YUAN CHEN, Hsinchu County (TW); Ren-Jeng Wang, Hsinchu County (TW)

(21) Appl. No.: 19/300,628

(22) Filed: Aug. 14, 2025

(30) Foreign Application Priority Data
Mar. 21, 2025 (TW) 114110660

(51) Int. Cl. A61B 17/70; A61B 17/00

(52) U.S. Cl. CPC A61B 17/7082; A61B 2017/00398; A61B 2017/00477

(57) ABSTRACT

A screw implantation device includes a driving motor, a central shaft, a transmission assembly, a guide pin assembly, a reducer assembly, and a pedicle screw assembly. The central shaft is driven by the driving motor. The transmission assembly includes a hollow transmission shaft. The hollow transmission shaft is sleeved around and arranged coaxially with the central shaft. The hollow transmission shaft is driven by the driving motor. The guide pin assembly is detachably assembled to the hollow transmission shaft. The reducer assembly is connected to the driving motor and the hollow transmission shaft. A hollow pedicle screw of the pedicle screw assembly is detachably assembled to the hollow transmission shaft and connected to the guide pin assembly.

### CROSS-REFERENCE TO RELATED PATENT APPLICATION

This application claims the benefit of priority to Taiwan Patent Application No. 114110660, filed on March 21, 2025. The entire content of the above identified application is incorporated herein by reference.

### FIELD OF THE DISCLOSURE

The present disclosure relates to a screw implantation device, and more particularly to a screw implantation device for simplifying the steps of pedicle screw implantation.

### BACKGROUND OF THE DISCLOSURE

Spinal fusion surgery is a common procedure for treating low back pain, and involves repeating several steps: making a small incision on the skin surface, creating a guide hole by using instruments such as a trocar or bone drill, and then inserting a guide wire. Afterwards, a hollow pedicle screw is locked into the spine along the guide wire to complete the implantation and stabilization of the screws.

However, in general, drilling a guide hole requires the drill bit to rotate at a high rotational speed with a low torque, whereas the insertion of the hollow pedicle screw requires the screw to rotate at a low rotational speed with a high torque. Therefore, it needs to use different instruments in these procedures. In other words, the existing methods for implanting the pedicle screws require complicated surgical steps and exchange of instruments, which may induce the surgeon to omit necessary steps and result in positional deviation due to frequent instrument changes (i.e., poor drilling alignment accuracy), thereby causing a final implantation position to deviate from the initially predetermined location.

Therefore, how to overcome the above-mentioned problem through an improvement in structural design has become an important issue to be addressed in the related art.

### SUMMARY OF THE DISCLOSURE

In response to the above-referenced technical inadequacy, the present disclosure provides a screw implantation device to simplify the pedicle screw implantation procedure and address the issue of complicated surgical steps and the excessive number of instruments required in existing techniques.

In order to solve the above-mentioned problems, one of the technical aspects adopted by the present disclosure is to provide a screw implantation device, which includes a driving motor, a central shaft, a transmission assembly, a guide pin assembly, a reducer assembly, and a pedicle screw assembly. The central shaft is driven by the driving motor. The transmission assembly includes a hollow transmission shaft. The hollow transmission shaft is sleeved around and arranged coaxially with the central shaft. The hollow transmission shaft is driven by the driving motor. The hollow transmission shaft has a first end and a second end. The driving motor is disposed at the second end. The guide pin assembly is detachably assembled to the first end. The guide pin assembly includes a socket member, a coupling member, and a guide pin. The socket member is connected to the central shaft. The coupling member has a hollow structure. The coupling member is detachably connected to the socket member. The guide pin passes through and is connected to the socket member. The hollow transmission shaft is sleeved around the socket member and is connected to the coupling member. The reducer assembly is disposed at the second end of the hollow transmission shaft. The reducer assembly is connected to the driving motor and the hollow transmission shaft. The pedicle screw assembly includes a hollow pedicle screw. The pedicle screw assembly is detachably assembled to the first end of the hollow transmission shaft and is connected to the guide pin assembly.

In the screw implantation device provided by the present disclosure, the hollow transmission shaft is driven by the driving motor, and the guide pin assembly and the driving motor are respectively disposed at opposite ends of the hollow transmission shaft. Therefore, the length of the guide pin can be reduced to avoid bending or deformation caused by external forces, thereby improving the drilling alignment accuracy of the guide pin.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a screw implantation device according to the present disclosure;

FIG. 2 is a schematic exploded view of the screw implantation device according to the present disclosure;

FIG. 3 is a schematic view of a transmission assembly of the screw implantation device according to the present disclosure;

FIG. 4 is a schematic view of a guide pin assembly of the screw implantation device according to the present disclosure;

FIG. 5 is a schematic cross-sectional view taken along line V-V of FIG. 4;

FIG. 6 is a schematic view of a pedicle screw assembly of the screw implantation device according to the present disclosure;

FIG. 7 is a schematic view of the guide pin assembly being assembled to the transmission assembly according to the present disclosure;

FIG. 8 is a schematic view of a retaining member being assembled to the transmission assembly according to the present disclosure;

FIG. 9 is a schematic view of the pedicle screw assembly being assembled to the transmission assembly and the retaining member according to the present disclosure; and

FIG. 10 is a partial schematic enlarged cross-sectional view of the screw implantation device according to the present disclosure.

### DETAILED DESCRIPTION OF THE EXEMPLARY EMBODIMENTS

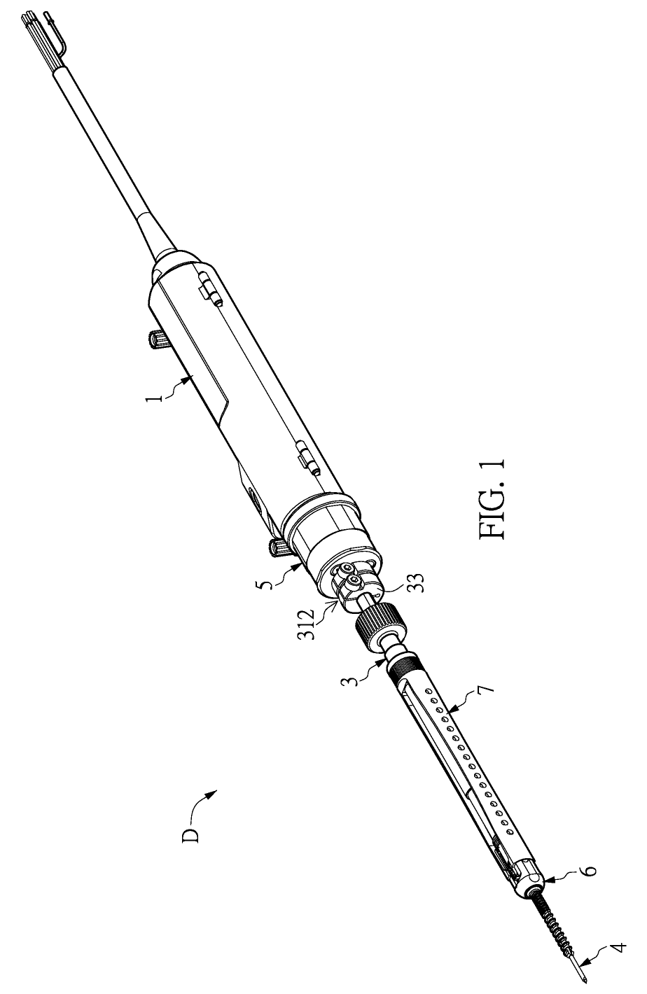

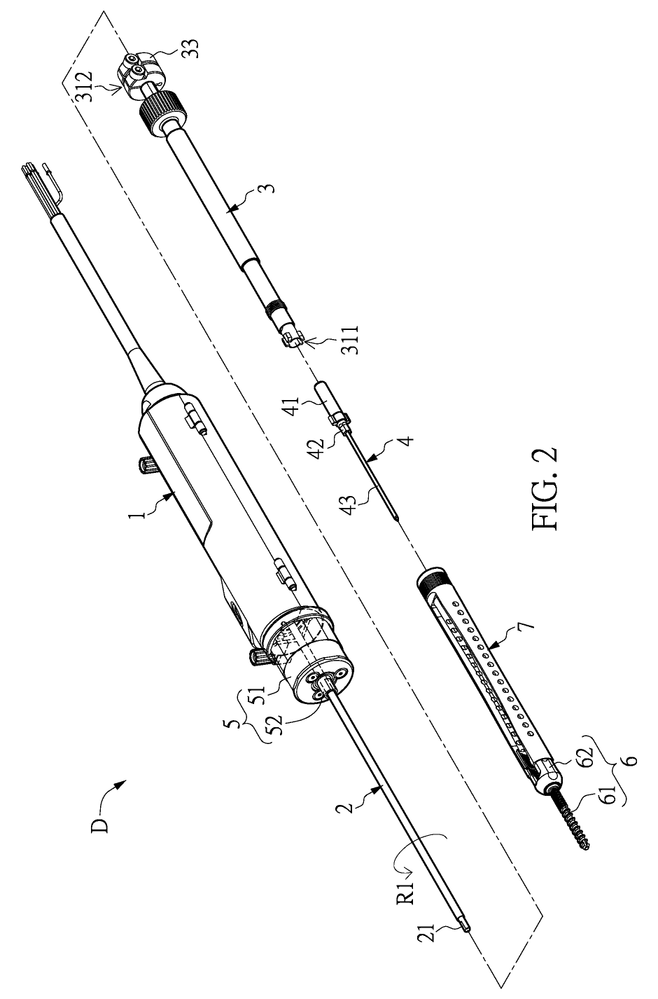

Referring to FIGS. 1 and 2, FIG. 1 is a schematic view of a screw implantation device according to the present disclosure, and FIG. 2 is a schematic exploded view of the screw implantation device according to the present disclosure. The present disclosure provides a screw implantation device D, which includes: a driving motor 1, a central shaft 2, a transmission assembly 3, a guide pin assembly 4, a reducer assembly 5, and a pedicle screw assembly 6. The central shaft 2 and the reducer assembly 5 are driven by the driving motor 1. The driving motor 1 is configured to output power to drive the central shaft 2 to rotate in a first rotational direction R1. The reducer assembly 5 includes a reducer 51 and an output shaft 52 that is connected to the reducer 51. The reducer 51 is connected to the driving motor 1. The reducer assembly 5 is used to connect the driving motor 1 and the transmission assembly 3.

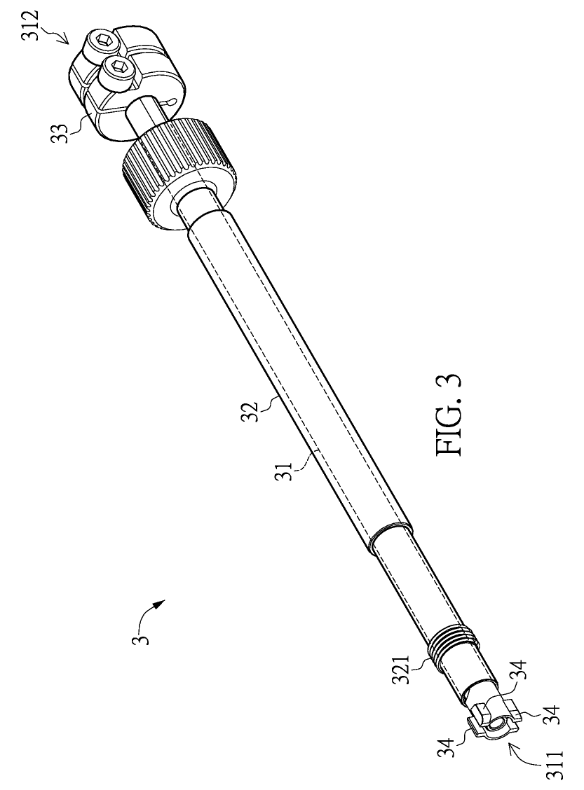

Reference is made to FIG. 3, which is a schematic view of a transmission assembly of the screw implantation device according to the present disclosure. The transmission assembly 3 includes a hollow transmission shaft 31 and a hollow connecting shaft 32. The hollow connecting shaft 32 is sleeved around the hollow transmission shaft 31. The hollow transmission shaft 31 has a first end 311 and a second end 312. The hollow transmission shaft 31 includes a fastening member 33 and a plurality of limiting members 34. The fastening member 33 is disposed at the second end 312, and the plurality of limiting members 34 are disposed at the first end 311. In addition, an outer wall of the hollow connecting shaft 32 is provided with external threads 321, the external threads 321 are adjacent to the first end 311.

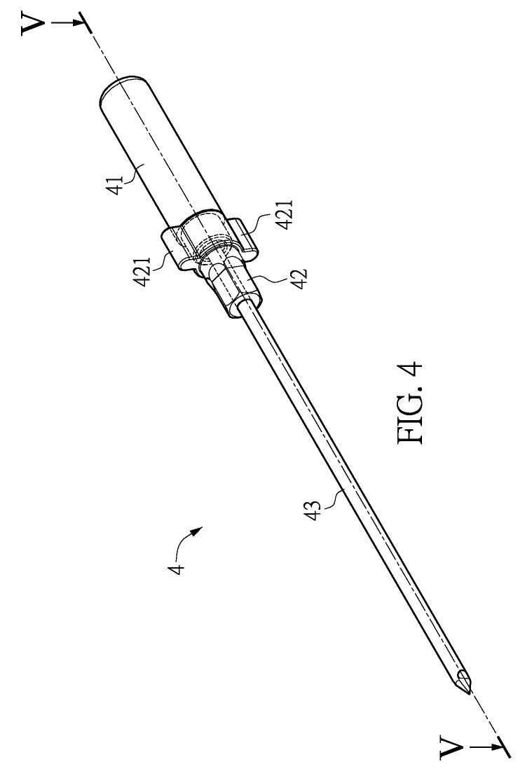

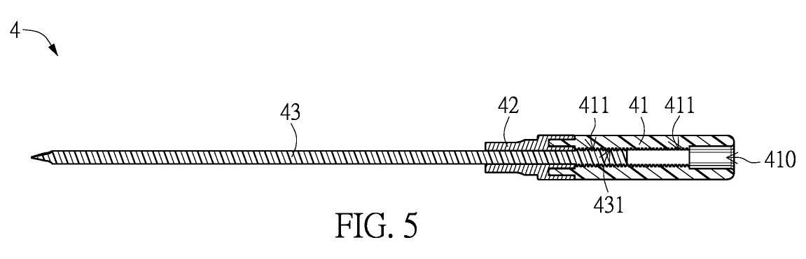

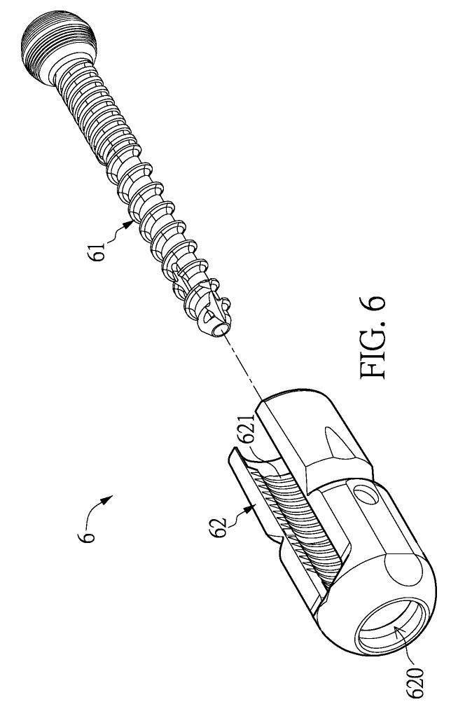

Reference is made to FIGS. 4 and 5. FIG. 4 is a schematic view of a guide pin assembly of the screw implantation device according to the present disclosure, and FIG. 5 is a schematic cross-sectional view taken along line V-V of FIG. 4. The guide pin assembly 4 includes a socket member 41, a coupling member 42, and a guide pin 43. The coupling member 42 has a hollow structure and includes two protruding portions 421. The two protruding portions 421 are disposed on opposite sides of the coupling member 42. The coupling member 42 is detachably connected to the socket member 41. The guide pin 43 passes through the coupling member 42 and is connected to the socket member 41. In addition, the socket member 41 includes a shaft hole 410 that is located on a side opposite to the coupling member 42. An inner wall of the socket member 41 is provided with internal threads 411, and a surface of the guide pin 43 is provided with external threads 431. The socket member 41 is threadedly engaged with the guide pin 43 through engagement between the internal threads 411 and the external threads 431. Referring to FIG. 6, which is a schematic view of a pedicle screw assembly of the screw implantation device according to the present disclosure. The pedicle screw assembly 6 includes a hollow pedicle screw 61 and a U-shaped connecting member 62. The U-shaped connecting member 62 has an opening 620. The hollow pedicle screw 61 passes through the opening 620 and is movably engaged with the U-shaped connecting member 62. It should be noted that spinning of the hollow pedicle screw 61 is not restricted by the U-shaped connecting member 62. Additionally, an inner wall of the U-shaped connecting member 62 is provided with internal threads 621.

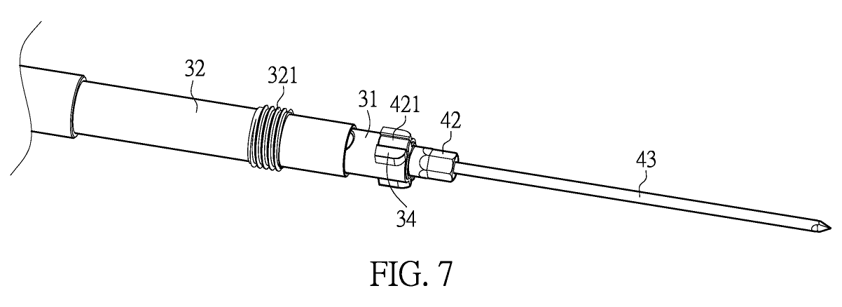

Reference is made to FIGS. 2 and 7. FIG. 7 is a schematic view of the guide pin assembly being assembled to the transmission assembly according to the present disclosure. The hollow transmission shaft 31 is sleeved around the central shaft 2, such that the driving motor 1 is disposed at the second end 312 of the hollow transmission shaft 31, and the hollow transmission shaft 31 is connected to the output shaft 52 of the reducer assembly 5 through the fastening member 33. In other words, the reducer assembly 5 is used to connect the driving motor 1 and the hollow transmission shaft 31. Specifically, the reducer 51 is connected to the driving motor 1, and the output shaft 52 is connected to the hollow transmission shaft 31. Accordingly, the hollow transmission shaft 31 and the central shaft 2 are coaxially arranged with each other, and the hollow transmission shaft 31 is driven by the driving motor 1. In addition, the guide pin assembly 4 is detachably assembled to the first end 311 of the hollow transmission shaft 31. That is, the guide pin assembly 4 is directly mounted to the front end of the screw implantation device D. The hollow transmission shaft 31 is sleeved around the socket member 41 and is connected to the coupling member 42. More specifically, each of the protruding portions 421 of the coupling member 42 is sandwiched between two limiting members 34.

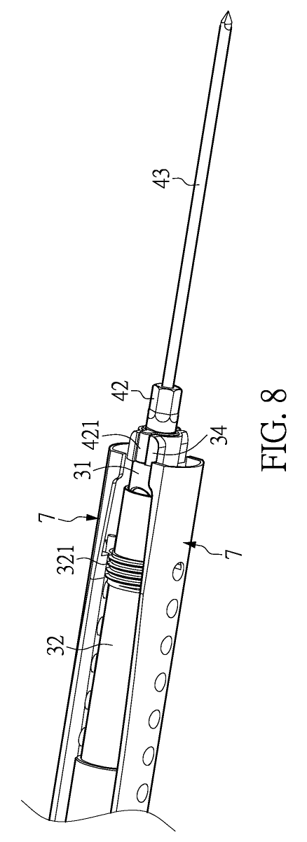

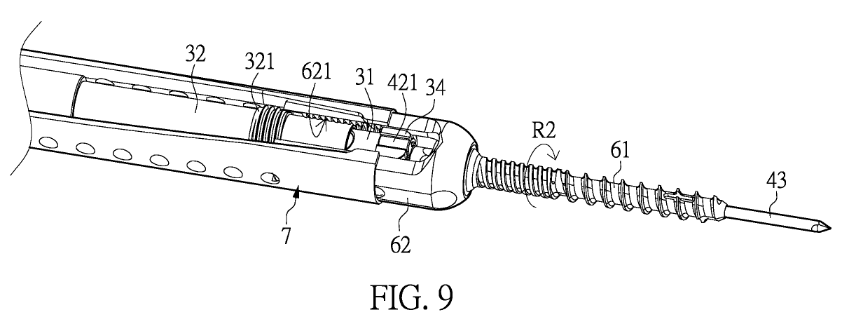

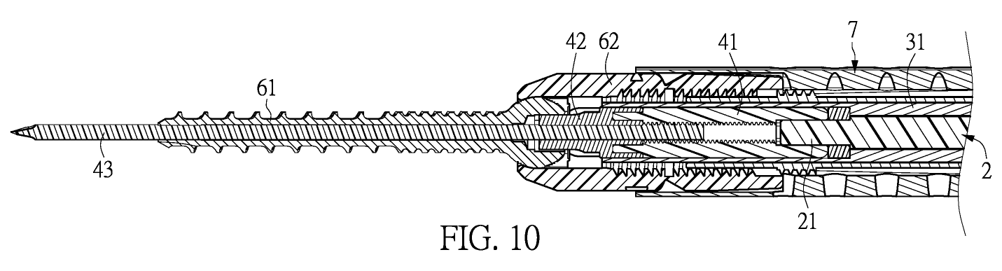

Reference is made to FIGS. 2 and 8. FIG. 8 is a schematic view of a retaining member being assembled to the transmission assembly according to the present disclosure. The screw implantation device D further includes a retaining member 7, which is sleeved around the transmission assembly 3. Reference is made to FIGS. 9 and 10. FIG. 9 is a schematic view of the pedicle screw assembly being assembled to the transmission assembly and the retaining member according to the present disclosure. FIG. 10 is a partial schematic enlarged cross-sectional view of the screw implantation device according to the present disclosure. The pedicle screw assembly 6 is connected to the guide pin assembly 4, and the pedicle screw assembly 6 and the guide pin assembly 4 are jointly assembled to the first end 311 of the hollow transmission shaft 31.

Specifically, as shown in FIG. 10, when the guide pin assembly 4 and the pedicle screw assembly 6 are assembled to the first end 311 of the hollow transmission shaft 31, the guide pin 43 passes through the hollow pedicle screw 61, and the coupling member 42 abuts against the hollow pedicle screw 61. As shown in FIGS. 2 and 6, a front end 21 of the central shaft 2 is inserted into the shaft hole 410 of the socket member 41, such that the central shaft 2, the socket member 41, and guide pin 43 are coaxially arranged with each other. Furthermore, as shown in FIG. 2, the output shaft 52 of the reducer assembly 5, the hollow transmission shaft 31, the coupling member 42 of the guide pin assembly 4, and the hollow pedicle screw 61 are coaxially arranged with each other.

In the related art, the guide pin used in a conventional screw implantation device is relatively long and must extend through the entire device for directly connecting to the driving motor, which is inconvenient for assembling or disassembling the implantation device. In contrast, in the screw implantation device D provided by the present disclosure, the guide pin 43 of the guide pin assembly 4 is relatively short and is indirectly connected to the driving motor 1 (i.e., the guide pin 43 is connected to the central shaft 2 through the socket member 41), which is more convenient for assembling or disassembling. Additionally, since the guide pin 43 is short, it is less likely to bend or deform the guide pin 43 by external forces during drilling, thereby reducing the risk of sliding or misalignment of the guide pin 43 and improving drilling alignment accuracy.

As shown in FIGS. 9 and 10, when the pedicle screw assembly 6 is assembled to the first end 311 of the hollow transmission shaft 31, the U-shaped connecting member 62 of the pedicle screw assembly 6 is connected to the retaining member 7. The hollow connecting shaft 32 is threadedly engaged with the U-shaped connecting member 62 through engagement between the internal threads 321 and the external threads 621, such that the hollow connecting shaft 32 is further connected to the pedicle screw assembly 6.

The following further recites operational steps of the screw implantation device D provided by the present disclosure, which mainly comprise a drilling step and a screw implantation step. As shown in FIGS. 2, 9, and 10, during the drilling step, the driving motor 1 drives the central shaft 2 to rotate in the first rotational direction R1 (see FIG. 2). Through the socket member 41, the central shaft 2 drives the guide pin 43 to move forward, and both the socket member 41 and the guide pin 43 rotate simultaneously in the first rotational direction R1. The guide pin 43 drills into a surgical site (e.g., a spine) at a high rotational speed with a low torque to form a pilot hole (not shown in the figures). Afterwards, the driving motor 1 stops driving the central shaft 2, thereby stopping the rotation of the central shaft 2 and the guide pin 43. Then, the driving motor 1 drives the reducer 51 through an internal one-way bearing (not shown in the figures), and the reducer 51 subsequently drives the hollow transmission shaft 31 to rotate in a second rotational direction R2 (see FIG. 9). Meanwhile, the hollow connecting shaft 32 remains stationary. The second rotational direction R2 is opposite to the first rotational direction R1. Through the configuration of the one-way bearing, the central shaft 2 can remain stationary when the driving motor 1 drives the hollow transmission shaft 31 in the reverse direction R2.

Afterwards, the hollow transmission shaft 31 drives the coupling member 42 and the hollow pedicle screw 61 to move forward and simultaneously rotate in the second rotational direction R2. Hence, the hollow pedicle screw 61 can be implanted into the patient’s body and screwed at the surgical site along the pilot hole at a low speed with a high torque (i.e., through the configuration of the reducer 51). It should be noted that during implantation of the hollow pedicle screw 61, the U-shaped connecting member 62 is still restricted by the retaining member 7 and the hollow connecting shaft 32 and remains fixed.

As shown in FIGS. 9 and 10, when the hollow transmission shaft 31 drives the coupling member 42 and the hollow pedicle screw 61 forward to screw into the surgical site, the socket member 41 that is connected to the coupling member 42 also rotates in the second rotational direction R2. However, since the guide pin 43 is leaning against the surgical site and does not rotate, the engagement between the internal threads 411 of the socket member 41 and the external threads 431 of the guide pin 43 causes the socket member 41 to rotate relative to the guide pin 43 in the opposite direction, thereby retracting the guide pin 43 from the surgical site into the socket member 41. In more detail, when the hollow pedicle screw 61 moves forward to engage with the surgical site, the guide pin 43 moves backward relative to the hollow pedicle screw 61 and retracts into the interior of the hollow pedicle screw 61.

### Beneficial Effects of the Embodiment

In the screw implantation device D provided by the present disclosure, the hollow transmission shaft 31 is driven by the driving motor 1, and the guide pin assembly 4 and the driving motor 1 are respectively disposed at opposite ends of the hollow transmission shaft 31. Therefore, the length of the guide pin 43 can be reduced to avoid bending or deformation caused by external forces, thereby improving the drilling alignment accuracy of the guide pin 43.

In the related art, the guide pin used in the conventional screw implantation device is relatively long and must extend through the entire device for directly connecting to the driving motor, which is inconvenient for assembling or disassembling the implantation device. In contrast, in the screw implantation device D provided by the present disclosure, the guide pin 43 of the guide pin assembly 4 is relatively short and is indirectly connected to the driving motor 1 (i.e., the guide pin 43 is connected to the central shaft 2 through the socket member 41), which is more convenient for assembling or disassembling. Additionally, since the guide pin 43 is short, it is less likely to bend or deform due to external forces during drilling, reducing the risk of sliding or misalignment of the guide pin 43 and improving drilling alignment accuracy.

Furthermore, in the screw implantation device D provided by the present disclosure, the guide pin 43 can drill into the surgical site (e.g., the spine) at a high rotational speed with a low torque to form the pilot hole, and then the hollow pedicle screw 61 can be implanted into the spine along the pilot hole and screwed at the surgical site at a low rotational speed with a high torque. In other words, the user can perform multiple steps (i.e., drilling and screw implantation) by just using the screw implantation device D, which not only reduces the number of times for changing instruments, but also simplifies the replacement procedures, thereby effectively improving surgical efficiency.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A screw implantation device, comprising:
a driving motor;
a central shaft driven by the driving motor;
a transmission assembly including a hollow transmission shaft, wherein the hollow transmission shaft is sleeved around and arranged coaxially with the central shaft, the hollow transmission shaft is driven by the driving motor, the hollow transmission shaft has a first end and a second end, and the driving motor is disposed at the second end;
a guide pin assembly detachably assembled to the first end of the hollow transmission shaft and including:
a socket member connected to the central shaft;
a coupling member having a hollow structure, wherein the coupling member is detachably connected to the socket member; and
a guide pin passing through and connected to the socket member;
wherein the hollow transmission shaft is sleeved around the socket member and is connected to the coupling member;
a reducer assembly disposed at the second end of the hollow transmission shaft, wherein the reducer assembly is connected to the driving motor and the hollow transmission shaft; and
a pedicle screw assembly including a hollow pedicle screw, wherein the pedicle screw assembly is detachably assembled to the first end of the hollow transmission shaft and is connected to the guide pin assembly.

2. The screw implantation device according to claim 1, wherein the reducer assembly includes a reducer and an output shaft that is connected to the reducer, the reducer is connected to the driving motor, and the output shaft is connected to the hollow transmission shaft.

3. The screw implantation device according to claim 2, wherein the output shaft, the hollow transmission shaft, the coupling member, and the hollow pedicle screw are arranged coaxially with each other.

4. The screw implantation device according to claim 1, wherein the central shaft, the socket member, and the guide pin are arranged coaxially with each other.

5. The screw implantation device according to claim 1, wherein the guide pin is indirectly connected to the driving motor.

6. The screw implantation device according to claim 1, wherein an inner wall of the socket member is provided with internal threads, a surface of the guide pin is provided with external threads, and the socket member is threadedly engaged with the guide pin through engagement between the internal threads and the external threads.

7. The screw implantation device according to claim 1, wherein the pedicle screw assembly further includes a U-shaped connecting member, the U-shaped connecting member has an opening, and the hollow pedicle screw passes through the opening and is movably engaged with the U-shaped connecting member.

8. The screw implantation device according to claim 7, wherein, when the guide pin assembly and the pedicle screw assembly are assembled to the first end of the hollow transmission shaft, the guide pin passes through the hollow pedicle screw, and the coupling member abuts against the hollow pedicle screw.

9. The screw implantation device according to claim 8, wherein the transmission assembly further includes a hollow connecting shaft, the hollow connecting shaft is sleeved around the hollow transmission shaft, an outer wall of the hollow connecting shaft is provided with external threads, and an inner wall of the U-shaped connecting member is provided with internal threads; wherein, when the guide pin assembly is assembled to the first end of the hollow transmission shaft, the hollow connecting shaft is threadedly engaged with the U-shaped connecting member through engagement between the internal threads and the external threads.

10. The screw implantation device according to claim 7, further comprising a retaining member that is sleeved around the transmission assembly and connected to the U-shaped connecting member.

11. The screw implantation device according to claim 1, wherein the driving motor drives the central shaft to rotate in a first rotational direction, such that the central shaft drives the socket member and the guide pin to move forward, and both the socket member and the guide pin simultaneously rotate in the first rotational direction.

12. The screw implantation device according to claim 11, wherein the driving motor drives the central shaft to rotate in a second rotational direction, such that the hollow transmission shaft drives the coupling member and the hollow pedicle screw to move forward, and both the coupling member and the hollow pedicle screw simultaneously rotate in the second rotational direction; wherein the second rotational direction is opposite to the first rotational direction.

13. The screw implantation device according to claim 12, wherein, when the hollow pedicle screw moves forward and simultaneously rotates in the second rotational direction, the guide pin moves backward relative to the hollow pedicle screw and retracts into the hollow pedicle.
